(19) United States
(12) Patent Application Publication
(10) Pub. No.: US 2010/0093764 A1
(43) Pub. Date: Apr. 15, 2010

(54) AMINES AND SULFOXIDES OF THIENO[2,3-d]PYRIMIDINE AND THEIR USE AS ADENOSINE A2a RECEPTOR ANTAGONISTS

(76) Inventors: Devraj Chakravarty, Hillsborough, NJ (US); Brian Christopher Shook, Gilbertsville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/499,483

(22) Filed: Jul. 8, 2009

Related U.S. Application Data
(60) Provisional application No. 61/104,796, filed on Oct. 13, 2008.

(51) Int. Cl. A61K 31/519; C07D 491/04; A61P 25/28

(52) U.S. Cl. 514/260.1; 544/278

(57) ABSTRACT

This invention relates to a novel thieno[2,3-d]pyrimidine, A, and its therapeutic and prophylactic uses, wherein R¹ and R² are defined in the specification. Disorders treated and/or prevented include Parkinson's Disease.

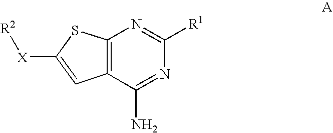

### CROSS-REFERENCE TO RELATED APPLICATIONS

The present application claims the benefits of the filing of U.S. Provisional Application No. 61/104,796 filed Oct. 13, 2008. The complete disclosures of the aforementioned related patent applications are hereby incorporated herein by reference for all purposes.

### FIELD OF THE INVENTION

This invention relates to a novel arylindenopyrimidine and its therapeutic and prophylactic uses. Disorders treated and/or prevented include neurodegenerative and movement disorders ameliorated by antagonizing Adenosine A2a receptors.

### BACKGROUND OF THE INVENTION

Adenosine A2a Receptors Adenosine is a purine nucleotide produced by all metabolically active cells within the body. Adenosine exerts its effects via four subtypes of cell surface receptors (A1, A2a, A2b and A3), which belong to the G protein coupled receptor superfamily (Stiles, G. L. Journal of Biological Chemistry, 1992, 267, 6451). A1 and A3 couple to inhibitory G protein, while A2a and A2b couple to stimulatory G protein. A2a receptors are mainly found in the brain, both in neurons and glial cells (highest level in the striatum and nucleus accumbens, moderate to high level in olfactory tubercle, hypothalamus, and hippocampus etc. regions) (Rosin, D. L.; Robeva, A.; Woodard, R. L.; Guyenet, P. G.; Linden, J. Journal of Comparative Neurology,1998, 401, 163).

In peripheral tissues, A2a receptors are found in platelets, neutrophils, vascular smooth muscle and endothelium (Gessi, S.; Varani, K.; Merighi, S.; Ongini, E.; Bores, P. A. British Journal of Pharmacology, 2000, 129, 2). The striatum is the main brain region for the regulation of motor activity, particularly through its innervation from dopaminergic neurons originating in the substantial nigra. The striatum is the major target of the dopaminergic neuron degeneration in patients with Parkinson's Disease (PD). Within the striatum, A2a receptors are co-localized with dopamine D2 receptors, suggesting an important site for the integration of adenosine and dopamine signaling in the brain (Fink, J. S.; Weaver, D. Ri; Rivkees, S. A.; Peterfreund, R. A.; Pollack, A. E.; Adler, E. M.; Reppert, S. M. Brain Research Molecular Brain Research, 1992, 14, 186).

Neurochemical studies have shown that activation of A2a receptors reduces the binding affinity of D2 agonist to their receptors. This D2R and A2aR receptor-receptorinteraction has been demonstrated instriatal membrane preparations of rats (Ferre, S.; con Euler, G.; Johansson, B.; Fredholm, B. B.; Fuxe, K. Proceedings of the National Academy of Sciences I of the United States of America, 1991, 88, 7238) as well as in fibroblast cell lines after transfected with A2aR and D2R cDNAs (Salim, H. ; Ferre, S.; Dalal, A.; Peterfreund, R. A.; Fuxe, K.; Vincent, J. D.; Lledo, P. M. Journal of Neurochemistry, 2000, 74, 432). In vivo, pharmacological blockade of A2a receptors using A2a antagonist leads to beneficial effects in dopaminergic neurotoxin MPTP(1-methyl-4-pheny-1,2,3,6-tetrahydropyridine)-induced PC) in various species, including mice, rats, and monkeys (Ikeda, K.; Kurokawa, M.; Aoyana, S.; Kuwana, Y. Journal of Neurochemistry, 2002, 80, 262).

Furthermore, A2a knockout mice with genetic blockade of A2a function have been found to be less sensitive to motor impairment and neurochemical changes when they were exposed to neurotoxin MPTP (Chen, J. F.; Xu, K.; I Petzer, J. P.; Steal, R.; Xu, Y. H.; Beilstein, M.; Sonsalla, P. K.; Castagnoli, K.; Castagnoli, N., Jr.; Schwarsschild, M. A. Journal of Neuroscience, 2001, 1 21, RC1 43).

In humans, the adenosine receptor antagonist theophylline has been found to produce beneficial effects in PD patients (Mally, J.; Stone, T. W. Journal of the Neurological Sciences, 1995, 132, 129). Consistently, recent epidemiological study has shown that high caffeine consumption makes people less likely to develop PD (Ascherio, A.; Zhang, S. M.; Heman, M. A.; Kawachi, I.; Colditz, G. A.; Speizer, F. E.; Willett, W. C. Annals of Neurology, 2001, 50, 56). In summary, adenosine A2a receptor blockers may provide a new class of antiparkinsonian agents (Impagnatiello, F.; Bastia, E.; Ongini, E.; Monopoli, A. Emerging Therapeutic Targets, 2000, 4, 635).

Antagonists of the A_{2A} receptor are potentially useful therapies for the treatment of addiction. Major drugs of abuse (opiates, cocaine, ethanol, and the like) either directly or indirectly modulate dopamine signaling in neurons particularly those found in the nucleus accumbens, which contain high levels of A_{2A} adenosine receptors. Dependence has been shown to be augmented by the adenosine signaling pathway, and it has been shown that administration of an A_{2A} receptor antagonist redues the craving for addictive substances (“The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316) and “Adaptations in Adenosine Signaling in Drug Dependence: Therapeutic Implications”, by Stephen P. Hack and Macdonald J. Christie, Critical Review in Neurobiology, Vol. 15, 235-274 (2003)). See also Alcoholism: Clinical and Experimental Research (2007), 31(8), 1302-1307.

An A_{2A} receptor antagonist could be used to treat attention deficit hyperactivity disorder (ADHD) since caffeine (a non selective adenosine antagonist) can be useful for treating ADHD, and there are many interactions between dopamine and adenosine neurons. Clinical Genetics (2000), 58(1), 31-40 and references therein.

Antagonists of the A_{2A} receptor are potentially useful therapies for the treatment of depression. A_{2A} antagonists are known to induce activity in various models of depression including the forced swim and tail suspension tests. The positive response is mediated by dopaminergic transmission and is caused by a prolongation of escape-directed behavior rather than by a motor stimulant effect. Neurology (2003), 61(suppl 6) S82-S87.

Antagonists of the A_{2A} receptor are potentially useful therapies for the treatment of anxiety. A_{2A} antagonist have been shown to prevent emotional/anxious responses in vivo. Neurobiology of Disease (2007), 28(2) 197-205.

### SUMMARY OF THE INVENTION

Compounds of Formula A are potent small molecule antagonists of the Adenosine A2a receptor.

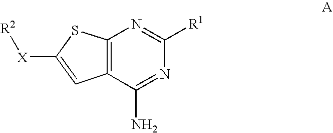

wherein:

R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, cyclopropyl and CN; or R¹ is heteroaryl optionally substituted with one substituent selected from the group consisting of: —OH, OC₍₁₋₄₎alkyl, CF₃, OCF₃, Cl, Br, —CN, F, CHF₂, cyclopropyl, and C₍₁₋₄₎alkyl;

R² is is an aromatic ring selected from the group consisting of phenyl and heteroaryl, wherein said aromatic ring is optionally substituted with —CN, F, Cl, Br, NO₂, —CF₃, OC₍₁₋₄₎alkyl, OCF₃, C₍₁₋₄₎alkyl, or cyclopropyl;

X is NH, NC₍₁₋₄₎alkyl, S, S(O), or S(O)₂;

and solvates, hydrates, tautomers, and pharmaceutically acceptable salts thereof.

### DETAILED DESCRIPTION OF THE INVENTION

The invention provides compounds of Formula A.

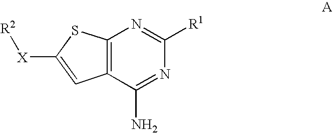

wherein:

R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, cyclopropyl and CN; or R¹ is heteroaryl optionally substituted with one substituent selected from the group consisting of: —OH, OC₍₁₋₄₎alkyl, CF₃, OCF₃, Cl, Br, —CN, F, CHF₂, cyclopropyl, and C₍₁₋₄₎alkyl;

R² is is an aromatic ring selected from the group consisting of phenyl and heteroaryl, wherein said aromatic ring is optionally substituted with —CN, F, Cl, Br, NO₂, —CF₃, OC₍₁₋₄₎alkyl, OCF₃, C₍₁₋₄₎alkyl, or cyclopropyl;

X is NH, NC₍₁₋₄₎alkyl, S, S(O), or S(O)₂;

and solvates, hydrates, tautomers, and pharmaceutically acceptable salts thereof.

In another embodiment of the invention:

R¹ is an aromatic ring selected from the group consisting of phenyl, furyl, oxazolyl, isoxazolyl, pyridyl, and thiazolyl, wherein said aromatic ring is optionally substituted with —CN, F, Cl, Br, —CF₃, OC₍₁₋₄₎alkyl, OCF₃, C₍₁₋₄₎alkyl, or cyclopropyl;

R² is is an aromatic ring selected from the group consisting of phenyl, furyl, oxazolyl, isoxazolyl, pyridyl, and thiazolyl, wherein said aromatic ring is optionally substituted with —CN, F, Cl, Br, NO₂, —CF₃, OC₍₁₋₄₎alkyl, OCF₃, C₍₁₋₄₎alkyl, or cyclopropyl;

X is NH, NC₍₁₋₄₎alkyl, S, S(O), or S(O)₂;

and solvates, hydrates, tautomers, and pharmaceutically acceptable salts thereof.

In another embodiment of the invention:

R¹ is an aromatic ring selected from the group consisting of phenyl, furyl, oxazolyl, isoxazolyl, pyridyl, and thiazolyl, wherein said aromatic ring is optionally substituted with —CN, F, —CF₃, OC₍₁₋₄₎alkyl, OCF₃, C₍₁₋₄₎alkyl, or cyclopropyl;

R² is an aromatic ring selected from the group consisting of phenyl, furyl, oxazolyl, isoxazolyl, pyridyl, and thiazolyl, wherein said aromatic ring is optionally substituted with —CN, F, Cl, —CF₃, OC₍₁₋₄₎alkyl, OCF₃, C₍₁₋₄₎alkyl, or cyclopropyl;

X is NH, NC₍₁₋₄₎alkyl, S, or S(O)₂;

and solvates, hydrates, tautomers, and pharmaceutically acceptable salts thereof.

In another embodiment of the invention:

R¹ is phenyl, wherein said phenyl is optionally substituted with —CN, —CF₃, OC₍₁₋₄₎alkyl, OCF₃, C₍₁₋₄₎alkyl, or cyclopropyl;

R² is phenyl, or pyridyl, wherein said phenyl, or pyridyl is optionally substituted with —CN, F, Cl, —CF₃, OC₍₁₋₄₎alkyl, OCF₃, C₍₁₋₄₎alkyl, or cyclopropyl;

X is NH, NC₍₁₋₄₎alkyl, or S(O)₂;

and solvates, hydrates, tautomers, and pharmaceutically acceptable salts thereof.

In another embodiment of the invention:

R¹ is phenyl, wherein said phenyl is substituted with —CN;

R² is phenyl, or pyridyl, wherein said phenyl, or pyridyl is optionally substituted with OCH₃, Cl, or F;

X is NH, NCH₃, or S(O)₂;

and solvates, hydrates, tautomers, and pharmaceutically acceptable salts thereof.

In another embodiment of the invention, the invention is directed to a compound selected from the group consisting of:

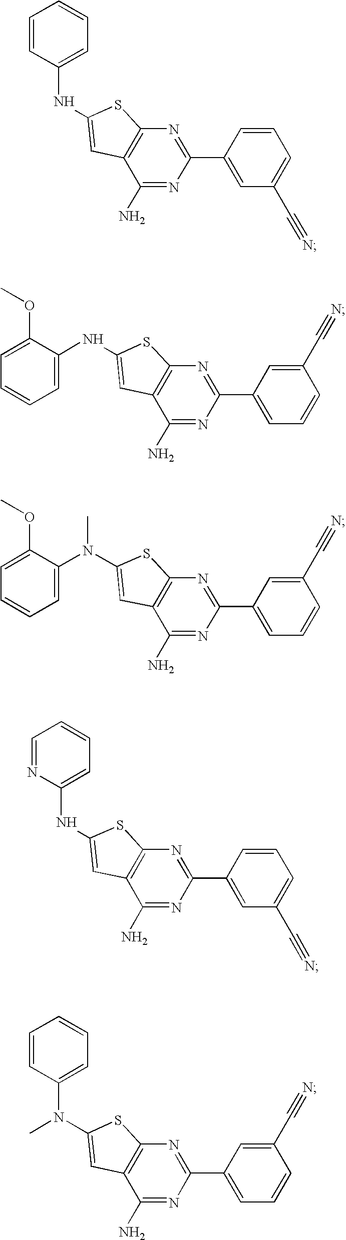

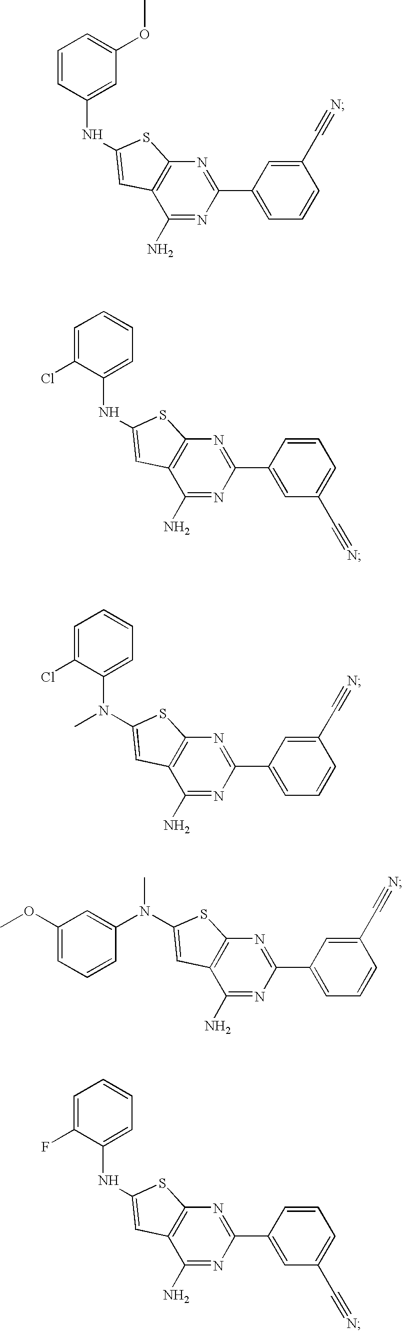

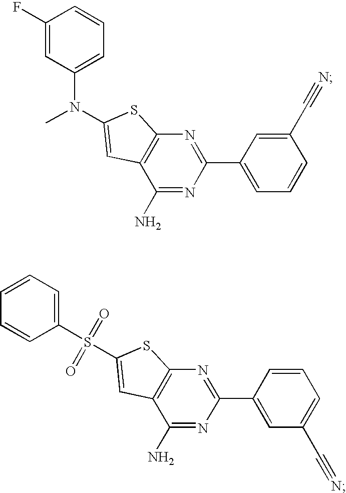

and solvates, hydrates, tautomers and pharmaceutically acceptable salts thereof.

This invention further provides a method of treating a subject having a condition ameliorated by antagonizing Adenosine A2a receptors, which comprises administering to the subject a therapeutically effective dose of a compound of Formula A.

This invention further provides a method of preventing a disorder ameliorated by antagonizing Adenosine A2a receptors in a subject, comprising of administering to the subject a prophylactically effective dose of the compound of claim 1 either preceding or subsequent to an event anticipated to cause a disorder ameliorated by antagonizing Adenosine A2a receptors in the subject.

Compounds of Formula A can be isolated and used as free bases. They can also be isolated and used as pharmaceutically acceptable salts.

Examples of such salts include hydrobromic, hydroiodic, hydrochloric, perchloric, sulfuric, maleic, fumaric, malic, tartaric, citric, adipic, benzoic, mandelic, methanesulfonic, hydroethanesulfonic, benzenesulfonic, oxalic, palmoic, 2 naphthalenesulfonic, p-toluenesulfonic, cyclohexanesulfamic and saccharinc

This invention also provides a pharmaceutical composition comprising a compound of Formula A and a pharmaceutically acceptable carrier.

Pharmaceutically acceptable carriers are well known to those skilled in the art and include, but are not limited to, from about 0.01 to about 0.1 M and preferably 0.05 M phosphate buyer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a condition ameliorated by antagonizing Adenosine A2a receptors, which comprises administering to the subject a therapeutically effective dose of a compound of Formula A.

In one embodiment, the disorder is a neurodegenerative or movement disorder. Examples of disorders treatable by the instant pharmaceutical composition include, without limitation, Parkinson's Disease, Huntington's Disease, Multiple System Atrophy, Corticobasal Degeneration, Alzheimer's Disease, and Senile Dementia.

In one preferred embodiment, the disorder is Parkinson's disease.

As used herein, the term “subject” includes, without limitation, any animal or artificially modified animal having a disorder ameliorated by antagonizing adenosine A2a receptors. In a preferred embodiment, the subject is a human.

Administering the instant pharmaceutical composition can be effected or performed using any of the various methods known to those skilled in the art. Compounds of Formula A can be administered, for example, intravenously, intramuscularly, orally and subcutaneously. In the preferred embodiment, the instant pharmaceutical composition is administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for the instant pharmaceutical composition. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of a compound of Formula A. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0, ug/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The invention also provides a method of treating addiction in a mammal, comprising administering a therapeutically effective dose of a compound of Formula A.

The invention also provides a method of treating ADHD in a mammal, comprising administering a therapeutically effective dose of a compound of Formula A.

The invention also provides a method of treating depression in a mammal, comprising administering a therapeutically effective dose of a compound of Formula A.

The invention also provides a method of treating anxiety in a mammal, comprising administering a therapeutically effective dose of a compound of Formula A.

Definitions:

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “heteroaryl” refers to a radical derived by the removal of one hydrogen atom from a ring carbon atom of a heteroaromatic ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

Abbreviations:

Herein and throughout this application, the following abbreviations may be used.

Cy cyclohexyl

DMF dimethylformamide

DMSO dimethylsulfoxide

Et ethyl

EtOAc ethyl acetate

KOtBu potassium tert-butoxide

Me methyl

NBS N-bromo succinimide

OAc acetate

Pd(dppf)Cl₁ [1,1′-Bis(diphenylphosphino)ferrocene]dichloropalladium (II)

py pyridine

THF tetrahydrofuran

Xantphos 9,9-Dimethyl-4,5-bis(diphenylphosphino)xanthene

### EXAMPLES

Compounds of formula A can be prepared by methods known to those who are skilled in the art. The following reaction schemes are only meant to represent examples of the invention and are in no way meant to be a limit of the invention.

Procedure

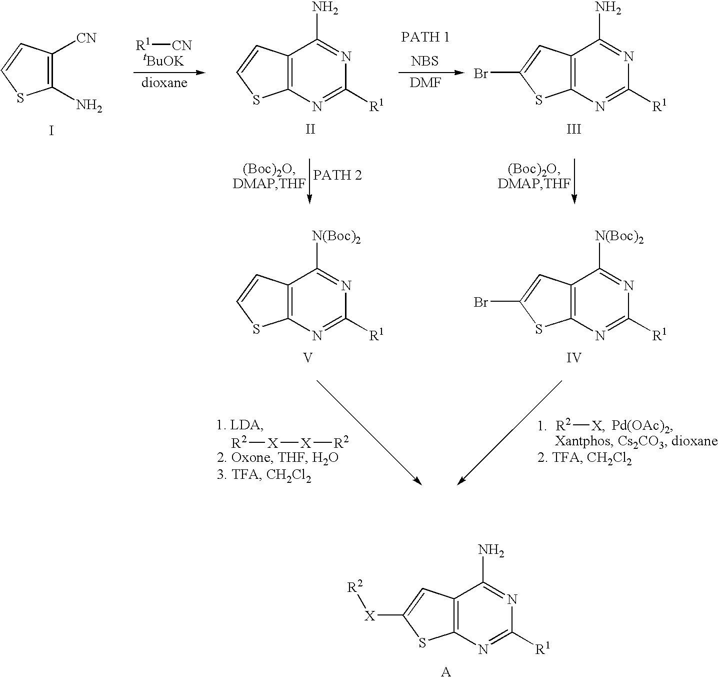

Scheme 1 illustrates the synthetic routes (Paths 1 and 2) leading to compounds of Formula A. Starting with 2-amino-3cyanothiophene I and following path 1 indicated by the arrows, condensation under basic conditions with arylnitriles affords the aminopyrimidine II. The aminopyrimidine II is then reacted with N-bromosuccinimide (NBS), which gives the bromothiophene III. Reacting the amino group in III with di-tert-butyldicarbonate [(Boc)₂O] in the presence of 4-dimethylamino pyridine (DMAP) gives the corresponding protected amine IV. Palladium catalyzed amination followed by trifluoroacetic acid (TFA) deprotection gives compounds of the formula A. Alternatively, following path 2, aminopyrimidine II can be reacted with (Boc)₂O in the presence of 4-dimethylamino pyridine (DMAP) gives the corresponding protected amine V. The thiophene V can be deprotonated with lithium diisopropylamide (LDA) and reacted with aryldisulfides to give an intermediate arylsulfide that is then oxidized to the corresponding sulfone with Oxone and finally deprotected with TFA to give compounds of the formula A.

### Example 1

### (4-Amino-6-phenylamino-thieno[2,3-d]pyrimidin-2-yl)-benzonitrile

### Example 1: Step a

### 3-(4-Amino-thieno[2,3-d]pyrimidin-2-yl)-benzonitrile

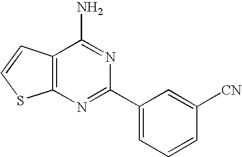

Solid potassium-tert-butoxide (1.1 g, 10.1 mmol) was added to a dioxane solution (20 mL) of 2-Amino-thiophene-3-carbonitrile (5.0 g, 40.3 mmol) and 1,3-dicyanobenzene (7.2 g, 56.5 mmol). The resulting slurry was stirred vigorously at 130° C. for 15 minutes. The dark slurry was cooled to room temperature, diluted with THF, and dry packed onto silica gel. The material was the purified via column chromatography to give 10.2 g of the title compound.

### Example 1: Step b

### 3-(4-Amino-6-bromo-thieno[2,3-d]pyrimidin-2-yl)-benzonitrile

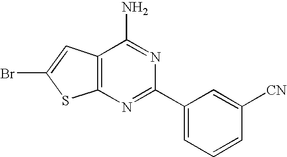

Solid NBS (1.6 g, 8.7 mmol) was added to a DMF solution (20 mL) of 3-(4-Amino-thieno[2,3-d]pyrimidin-2-yl)-benzonitrile (2.0 g, 7.9 mmol). After 45 minutes water was added and the resulting precipitate was collected by filtration, washed with water, and dried in vacuo to give 2.4 g of the title compound.

### Example 1: Step c

### [6-Bromo-2-(3-cyano-phenyl)-thieno[2,3-d]pyrimidin-4-yl]-bis-carbamic acid tert-butyl ester

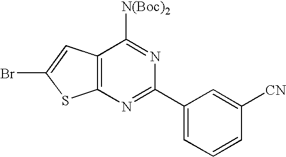

Solid DMAP (9 mg, 0.07 mmol) was added to a THF solution (4 mL) of 3-(4-Amino-6-bromo-thieno[2,3-d]pyrimidin-2-yl)-benzonitrile (240 mg, 0.73 mmol) and (Boc)₂O (396 mg, 1.81 mmol). After 16 h the mixture was diluted with EtOAc and then washed consecutively with water and brine, dried (Na₂SO₄), concentrated and purified via column chromatography to give 216 mg of the title compound.

### Example 1: Step d

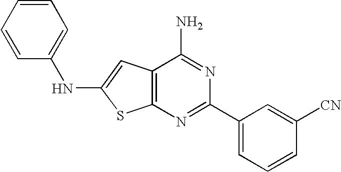

A dioxane solution (1.5 mL) of [6-Bromo-2-(3-cyano-phenyl)-thieno[2,3-d]pyrimidin-4-yl]-bis-carbamic acid tert-butyl ester (100 mg, 0.19 mmol), aniline (25 μL, 0.28 mmol), Xantphos (11 mg, 0.02 mmol), Pd(OAc)₂ (4 mg, 0.02 mmol), and Cs₂CO₃ (142 mg, 0.44 mmol) was heated to 100° C. After 4 h the mixture was cooled to rt, diluted with EtOAc and washed with water and brine, dried (Na₂SO₄), concentrated and purified via column chromatography to give 56 mg of the amine coupled product. This material was then dissolved in CH₂Cl₂/TFA (2 mL/0.5 mL) and stirred at room temperature. After 2 h the solution was neutralized with saturated aqueous NaHCO₃ and the aqueous layer was extracted with EtOAc, dry packed onto silica gel and then purified via column chromatography to give 33 mg of the title compound as the free base, which was dissolved in THF and added to 1 mL of 1 N HCl in ether, concentrated, and dried in vacuo to give the title compound as the di-HCl salt. ¹H NMR (CHLOROFORM-d, 300 MHz): δ=8.75 (s, 1H), 8.66 (d, J=7.9 Hz, 1H), 7.70 (d, J=7.5 Hz, 1H), 7.57 (t, J=7.9 Hz, 1H), 7.29-7.39 (m, 3H), 7.17-7.24 (m, 2H), 7.04-7.15 (m, 1H), 6.52 (s, 1H), 6.39 ppm (s, 1H) 3.17-3.80 ppm (m, 1H); MS m/e 344 (M+H).

### Example 2

### 3-[4-Amino-6-(2-methoxy-phenylamino)-thieno[2,3-d]pyrimidin-2-yl]-benzonitrile

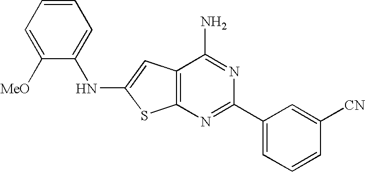

The title compound was prepared using 2-methoxy-phenylamine in place of aniline as described in Example 1. ¹H NMR (CHLOROFORM-d, 300 MHz): δ=8.74 (s, 1H), 8.65 (d, J=7.9 Hz, 1H), 7.68 (d, J=7.5 Hz, 1H), 7.54 (t, J=7.7 Hz, 1H), 7.34-7.45 (m, 1H), 6.87-7.01 (m, 3H), 6.67 (s, 1H), 6.61 (s, 1H), 5.16 (br. s., 2H), 3.93 ppm (s, 3H); MS m/e 374 (M+H).

### Example 3

### 3-{4-Amino-6-[(2-methoxy-phenyl)-methyl-amino]-thieno[2,3-d]pyrimidin-2-yl}-benzonitrile

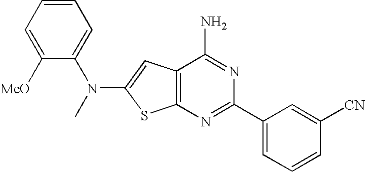

The title compound was prepared using (2-methoxy-phenyl)-methyl-amine in place of aniline as described in Example 1. ¹H NMR (CHLOROFORM-d, 300 MHz): δ=8.67 (s, 1H), 8.58 (d, J=7.9 Hz, 1H), 7.63 (d, J=7.9 Hz, 1H), 7.50 (t, J=7.9 Hz, 1H), 7.30-7.39 (m, 2H), 6.96 -7.07 (m, 2H), 5.70 (s, 1H), 5.00 (br. s., 2H), 3.84 (s, 3H), 3.35 ppm (s, 3H); MS m/e 388 (M+H).

### Example 4

### 3-[4-Amino-6-(pyridin-2-ylamino)-thieno[2,3-d]pyrimidin-2-yl]-benzonitrile

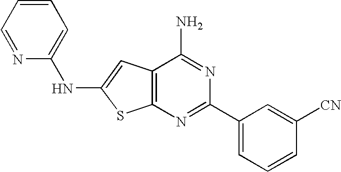

The title compound was prepared using pyridin-2-ylamine in place of aniline as described in Example 1. ¹H NMR (Acetone, 300 MHz): δ=9.58 (s, 1H), 8.45-8.77 (m, 2H), 8.19 (d, J=4.1 Hz, 1H), 7.67 (d, J=7.5 Hz, 1H), 7.45-7.59 (m, 2H), 6.69-6.87 (m, 3H), 6.50 ppm (br. s., 2 H); MS m/e 345 (M+H).

### Example 5

### 3-[4-Amino-6-(methyl-phenyl-amino)-thieno[2,3-d]pyrimidin-2-yl]-benzonitrile

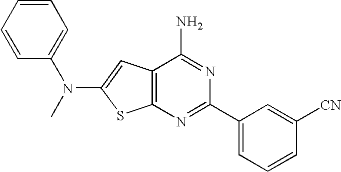

The title compound was prepared using N-methylaniline in place of aniline as described in Example 1. ¹H NMR (CHLOROFORM-d, 300 MHz): δ=8.70 (s, 1H), 8.60 (d, J=8.3 Hz, 1H), 7.65 (d, J=7.9 Hz, 1H), 7.52 (t, J=7.7 Hz, 1H), 7.35-7.45 (m, 2H), 7.23-7.33 (m, 3H), 7.12-7.23 (m, 1H), 5.14 (br. s., 2H), 3.45 ppm (s, 3H); MS m/e 358 (M+H).

### Example 6

### 3-[4-Amino-6-(3-methoxy-phenylamino)-thieno[2,3-d]pyrimidin-2-yl]-benzonitrile-hydrochloride

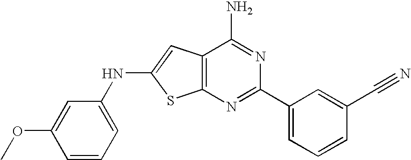

The title compound was prepared using 3-methoxy-phenylamine in place of aniline as described in Example 1. ¹H NMR (DMSO-d₆, 300 MHz): δ=8.55-8.72 (m, 2H), 7.94 (d, J=7.9 Hz, 1H), 7.72 (t, J=7.9 Hz, 2H), 7.16-7.29 (m, 1H), 7.06 (s, 1H), 6.71-6.83 (m, 2H), 6.47-6.59 (m, 1H), 3.76 ppm (s, 3H); MS m/e 374 (M+H).

### Example 7

### 3-[4-Amino-6-(2-chloro-phenylamino)-thieno[2,3-d]pyrimidin-2-yl]-benzonitrile-hydrochloride

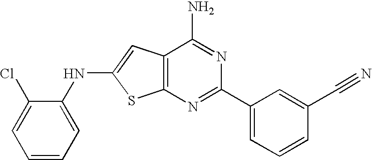

The title compound was prepared using 2-chloro-phenylamine in place of aniline as described in Example 1. ¹H NMR (DMSO-d₆, 300 MHz): δ=8.64 (s, 2H), 7.96 (s, 1H), 7.60-7.85 (m, 2H), 7.46 (s, 1H), 7.38 (s, 1H), 7.18-7.31 (m, 2H), 6.66 (s, 1H), 3.17 ppm (s, 2H); MS m/e 378 (M+H).

### Example 8

### 3-{4-Amino-6-[(2-chloro-phenyl)-m ethyl-amino]-thieno[2,3-d]pyrimidin-2-yl}-benzonitrile

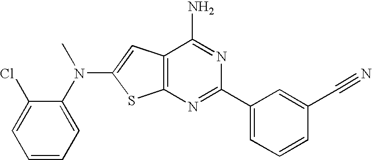

The title compound was prepared using (2-chloro-phenyl)-methyl-amine in place of aniline as described in Example 1. ¹H NMR (DMSO-d₆, 300 MHz): δ=8.66 (s, 1H), 8.57 (s, 1H), 7.99 (s, 1H), 7.67-7.77 (m, 2H), 7.62 (d, J=1.9 Hz, 1H), 7.45-7.51 (m, 2H), 7.31 (s, 1H), 6.56 (s, 2H), 3.37 ppm (s, 3H); MS m/e 392 (M+H).

### Example 9

### 3-{4-Amino-6-[(3-methoxy-phenyl)-methyl-amino]-thieno[2,3-d]pyrimidin-2-yl}-benzonitrile-hydrochloride

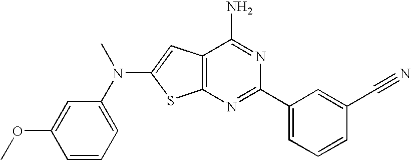

The title compound was prepared using (3-methoxy-phenyl)-methyl-amine in place of aniline as described in Example 1. ¹H NMR (DMSO-d₆, 300 MHz): δ=8.51-8.66 (m, 2H), 7.93 (d, J=7.5 Hz, 1H), 7.71 (d, J=7.9 Hz, 1H), 7.29 (d, J=7.9 Hz, 1H), 6.82-6.94 (m, 4H), 6.73 (d, J=2.3 Hz, 1H), 3.76 (s, 3H), 3.40 ppm (s, 3H); MS m/e 388 (M+H).

### Example 10

### 3-[4-Amino-6-(2-fluoro-phenylamino)-thieno[2,3-d]pyrimidin-2-yl]-benzonitrile

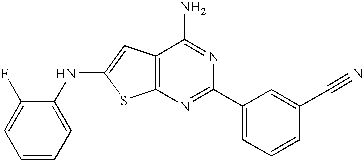

The title compound was prepared using 2-fluoro-phenylamine in place of aniline as described in Example 1. ¹H NMR (DMSO-d₆, 300 MHz): δ=9.17 (br. s., 1H), 8.52-8.75 (m, 2H) 7.96 (d, J=7.9 Hz, 1H), 7.62-7.82 (m, 2H), 6.93-7.33 ppm (m, 6H); MS m/e 362 (M+H).

### Example 11

### 3-{4-Amino-6-[(3-fluoro-phenyl)-methyl-amino]-thieno[2,3-d]pyrimidin-2-yl}-benzonitrile

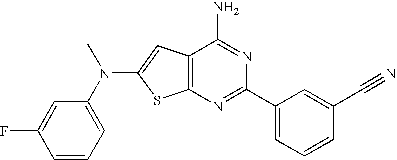

The title compound was prepared using (3-fluoro-phenyl)-methyl-amine in place of aniline as described in Example 1. ¹H NMR (CHLOROFORM-d, 300 MHz): δ=8.73 (s, 1H), 8.64 (d, J=7.9 Hz, 1H), 7.68 (d, J=7.5 Hz, 1H), 7.54 (t, J=7.9 Hz, 1H), 7.30 (d, J=6.8 Hz, 1H), 6.86-7.03 (m, 2H), 6.79 (s, 1H), 6.38 (s, 1H), 5.18 (br. s., 2H), 3.45 ppm (s, 3H); MS m/e 376 (M+H).

### Example 12

### 3-(4-Amino-6-benzenesulfonyl-thieno[2,3-d]pyrimidin-2-yl)-benzonitrile

### Example 12: Step a

### [2-(3-Cyano-phenyl)-thieno[2,3-d]pyrimidin-4-yl]-bis-carbamic acid tert-butyl ester

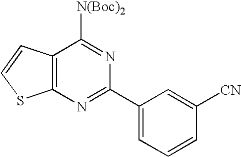

Solid DMAP (42 mg, 0.3 mmol) was added to a THF solution (17 mL) of 3-(4-Amino-thieno[2,3-d]pyrimidin-2-yl)-benzonitrile(850 mg, 3.4 mmol) and (Boc)₂O (1.8 g, 8.4 mmol). After 4 h the mixture was diluted with EtOAc and then washed consecutively with water and brine, dried (Na₂SO₄), concentrated and purified via column chromatography to give 1.2 g of the title compound.

### Example 12: Step b

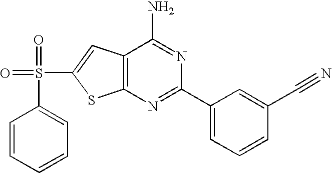

A 1.8 M LDA solution (0.675 ml, 1.2 mmol) was added dropwise a −78° C. THF solution (5 mL) of [2-(3-Cyano-phenyl)-thieno[2,3-d]pyrimidin-4-yl]-bis-carbamic acid tert-butyl ester (0.5 g, 1.1 mmol). After 5 minutes a THF solution (2 ml) of diphenyldisulfide (361 mg, 1.6 mmol) was added. After 30 minutes 10 ml of saturated aqueous NH₄Cl solution was added and the aqueous layer was extracted with EtOAc, dry packed onto silica gel and then purified via column chromatography to give 371 mg of the phenylsulfide adduct. Solid Oxone (0.986 g, 1.6 mmol) was added to 50:50 THF/water solution (6 mL) of the phenylsulfide compound (300 mg, 0.53 mmol). After 8 hours the mixture was diluted with EtOAc and then washed consecutively with saturated aqueous NaHCO₃ solution, water and brine, dried (Na₂SO₄), concentrated and used without further purification. This material was dissolved in a 50:50 CH₂Cl₂/TFA (6 mL) and stirred. After 30 minutes the solution was neutralized with saturated aqueous NaHCO₃ solution and the aqueous layer was extracted with EtOAc, dry packed onto silica gel and then purified via column chromatography to give 200 mg of of the title. ¹H NMR (DMSO-d₆, 300 MHz): δ=8.61 (s, 1H), 8.50 (s, 1H), 8.21 (br. s., 2H), 7.95-8.05 (m, 3H), 7.65-7.82 ppm (m, 5H); MS m/e 393 (M+H).

### Biological Assays and Activity

### Ligand Binding Assay for Adenosine A2a Receptor

Ligand binding assay of adenosine A2a receptor was performed using plasma membrane of HEK293 cells containing human A2a adenosine receptor (PerkinElmer, RB-HA2a) and radioligand [³H]CGS21680 (PerkinElmer, NET1021). Assay was set up in 96-well polypropylene plate in total volume of 200 μL by sequentially adding 20 μL1:20 diluted membrane, 130 μL assay buffer (50 mM Tri•HCl, pH7.4 10 mM MgCl₂, 1 mM EDTA) containing [³H] CGS21680, 50 μL diluted compound (4×) or vehicle control in assay buffer. Nonspecific binding was determined by 80 mM NECA. Reaction was carried out at room temperature for 2 hours before filtering through 96-well GF/C filter plate pre-soaked in 50 mM Tris•HCl, pH7.4 containing 0.3% polyethylenimine. Plates were then washed 5 times with cold 50 mM Tris•HCl, pH7.4, dried and sealed at the bottom. Microscintillation fluid 30 μL was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693)

### Adenosine A2a Receptor Functional Assay (A2AGAL2)

To initiate the functional assay, cryopreserved CHO—K1 cells overexpressing the human adenosine A2a receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 15 nM NECA (Sigma E2387) agonist challenge (5 uL volume). A control curve of NECA, a DMSO/Media control, and a single dose of Forskolin (Sigma F3917) were also included on each plate. After additions, cell plates were allowed to incubate at 37° C., 5% CO₂, 90% Rh for 5.5-6 hours. After incubation, media was removed, and cell plates were washed 1× 50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

### Adenosine A1 Receptor Functional Assay (A1GAL2)

To initiate the functional assay, cryopreserved CHO—K1 cells overexpressing the human adenosine A1 receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 4 nM r-PIA (Sigma P4532)/1 uM Forskolin (Sigma F3917) agonist challenge (5 uL volume). A control curve of r-PIA in 1 uM Forskolin, a DMSO/Media control, and a single dose of Forskolin were also included on each plate. After additions, cell plates were allowed to incubate at 37° C., 5% CO₂, 90% Rh for 5.5-6 hours. After incubation, media was removed, and cell plates were washed 1× 50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

A2a ASSAY DATA

| Example | A2AGAL2 Ki (uM) | A1GAL2 Ki (uM) |
|---|---|---|
| 1 | 0.0893923 | 1.14895 |
| 2 | 0.0113527 | 0.268968 |
| 3 | 0.102825 | 0.569115 |
| 4 | 0.0843529 | 0.765421 |
| 5 | 0.12659 | 2.33185 |
| 6 | 0.261337 | 2.37246 |
| 7 | 0.23 | 2.27405 |
| 8 | 0.316228 | 2.77268 |
| 9 | 0.273024 | 2.5363 |
| 10 | 0.13116 | 2.59777 |
| 11 | 0.398382 | 5.22998 |
| 12 | 0.0157362 | 0.358344 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

1. The compounds of Formula A. 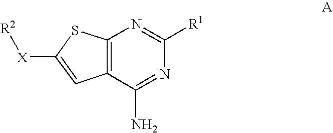
wherein:
R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, cyclopropyl and CN; or R¹ is heteroaryl optionally substituted with one substituent selected from the group consisting of: —OH, OC₍₁₋₄₎alkyl, CF₃, OCF₃, Cl, Br, —CN, F, CHF₂, cyclopropyl, and C₍₁₋₄₎alkyl;
R² is an aromatic ring selected from the group consisting of phenyl and heteroaryl, wherein said aromatic ring is optionally substituted with —CN, F, Cl, Br, NO₂, —CF₃, OC₍₁₋₄₎alkyl, OCF₃, C₍₁₋₄₎alkyl, or cyclopropyl;
X is NH, NC₍₁₋₄₎alkyl, S, S(O), or S(O)₂;
and solvates, hydrates, tautomers, and pharmaceutically acceptable salts thereof.

2. A compound of claim 1 wherein:
R¹ is an aromatic ring selected from the group consisting of phenyl, furyl, oxazolyl, isoxazolyl, pyridyl, and thiazolyl, wherein said aromatic ring is optionally substituted with —CN, F, Cl, Br, —CF₃, OC₍₁₋₄₎alkyl, OCF₃, C₍₁₋₄₎alkyl, or cyclopropyl;
R² is an aromatic ring selected from the group consisting of phenyl, furyl, oxazolyl, isoxazolyl, pyridyl, and thiazolyl, wherein said aromatic ring is optionally substituted with —CN, F, Cl, Br, NO₂, —CF₃, OC₍₁₋₄₎alkyl, OCF₃, C₍₁₋₄₎alkyl, or cyclopropyl;
and solvates, hydrates, tautomers, and pharmaceutically acceptable salts thereof.

3. A compound of claim 2 wherein:
R¹ is an aromatic ring selected from the group consisting of phenyl, furyl, oxazolyl, isoxazolyl, pyridyl, and thiazolyl, wherein said aromatic ring is optionally substituted with —CN, F, —CF₃, OC₍₁₋₄₎alkyl, OCF₃, C₍₁₋₄₎alkyl, or cyclopropyl;
R² is an aromatic ring selected from the group consisting of phenyl, furyl, oxazolyl, isoxazolyl, pyridyl, and thiazolyl, wherein said aromatic ring is optionally substituted with —CN, F, Cl, —CF₃, OC₍₁₋₄₎alkyl, OCF₃, C₍₁₋₄₎alkyl, or cyclopropyl;
and solvates, hydrates, tautomers, and pharmaceutically acceptable salts thereof.

4. A compound of claim 3 wherein:
R¹ is phenyl, wherein said phenyl is optionally substituted with —CN, —CF₃, OC₍₁₋₄₎alkyl, OCF₃, C₍₁₋₄₎alkyl, or cyclopropyl;
R² is phenyl, or pyridyl, wherein said phenyl, or pyridyl is optionally substituted with —CN, F, Cl, —CF₃, OC₍₁₋₄₎alkyl, OCF₃, C₍₁₋₄₎alkyl, or cyclopropyl;
X is NH, NC₍₁₋₄₎alkyl, or S(O)₂;
and solvates, hydrates, tautomers, and pharmaceutically acceptable salts thereof.

5. A compound of claim 4 wherein:
R¹ is phenyl, wherein said phenyl is substituted with —CN;
R² is phenyl, or pyridyl, wherein said phenyl, or pyridyl is optionally substituted with OCH₃, Cl, or F;
X is NH, NCH₃, or S(O)₂;
and solvates, hydrates, tautomers, and pharmaceutically acceptable salts thereof.

6. A compound selected from the group consisting of: 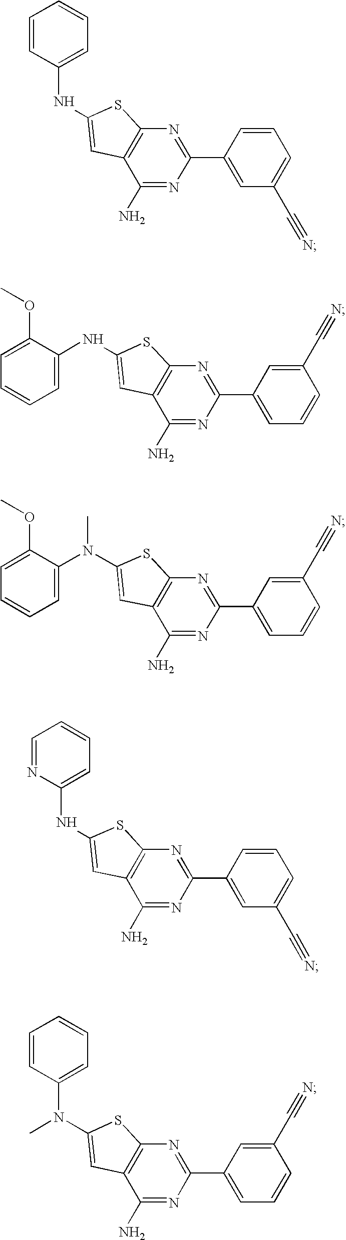 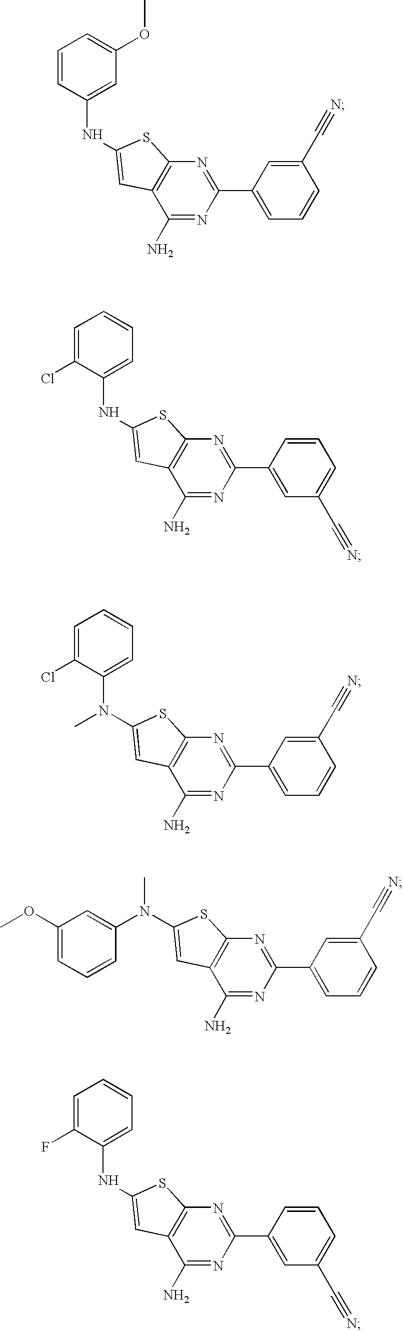 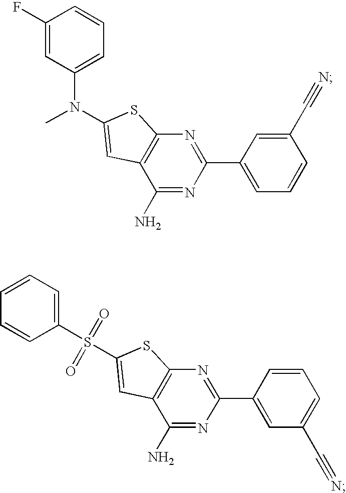
and solvates, hydrates, tautomers and pharmaceutically acceptable salts thereof.

7. A pharmaceutical composition comprising the compound of claim 1, and a pharmaceutically acceptable carrier.

8. A method of treating a subject having a disorder ameliorated by antagonizing Adenosine A2a receptors in appropriate cells in the subject, which comprises administering to the subject a therapeutically effective dose of the compound of claim 1.

9. A method of preventing a disorder ameliorated by antagonizing Adenosine A2a receptors in appropriate cells in the subject, comprising administering to the subject a prophylactically effective dose of the compound of claim 1, either preceding or subsequent to an event anticipated to cause a disorder ameliorated by antagonizing Adenosine A2a receptors in appropriate cells in the subject.

10. The method of treating a subject having a disorder ameliorated by antagonizing Adenosine A2a receptrors in appropriate cells in the subject, comprising administering to the subject a therapeutically or prophylactically effective dose of the pharmaceutical composition of claim 7.

11. The method of preventing a disorder ameliorated by antagonizing Adenosine A2a receptors in appropriate cells in the subject, comprising administering to the subject a therapeutically or prophylactically effective dose of the pharmaceutical composition of claim 7.

12. The method of claim 8, wherein the disorder is a neurodegenerative disorder or a movement disorder.

13. The method of claim 8, wherein the disorder is selected from the group consisting of Parkinson's Disease, Huntington's Disease, Multiple System Atrophy, Corticobasal Degeneration, Alzheimer's Disease, and Senile Dementia.

14. The method of claim 9, wherein the disorder is a neurodegenerative disorder or a movement disorder.

15. The method of claim 9, wherein the disorder is selected from the group consisting of Parkinson's Disease, Huntington's Disease, Multiple System Atrophy, Corticobasal Degeneration, Alzheimer's Disease, and Senile Dementia.

16. The method of claim 8, wherein the disorder is Parkinson's Disease.

17. The method of claim 8, wherein the disorder is addiction.

18. The method of claim 8, wherein the disorder is Attention Deficit Hyperactivity Disorder (ADHD).

19. The method of claim 8, wherein the disorder is depression.

20. The method of claim 8, wherein the disorder is anxiety.
